(19) United States
(12) Patent Application Publication
(10) Pub. No.: US 2008/0167679 A1
(43) Pub. Date: Jul. 10, 2008

(54) Cage and Sleeve Assembly for a Filtering Device

(76) Inventor: John E. Papp, Temecula, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/620,648

(22) Filed: Jan. 6, 2007

(51) Int. Cl. A61M 29/00; B21C 37/08

(52) U.S. Cl. 606/200; 29/34 R

(57) ABSTRACT

A cage and sleeve assembly for an embolic filtering device used to filter embolic particles from a body vessel has a strut assembly that is movable between an unexpanded position and an expanded position. Struts having strut ends at the respective ends form a cage. The strut ends are initially made from linear elastic nitinol, and a series of spot or laser or other types of welds then secure the strut ends in the sleeve assembly. In one approach, the ends of the strut ends are welded to form a tube. In another approach, the strut ends are welded onto a sleeve. The strut ends may optionally have ends that are partial cylinders, and the partial cylinders are welded onto a cylindrical sleeve. Effects from the welding, such as changing linear elastic nitinol to superelastic nitinol, are contained within a heat-effected zone, and do not extend into areas of the structure that typically bend during use.

### BACKGROUND OF THE INVENTION

The present invention relates generally to filtering devices used when an interventional procedure is being performed in a stenosed or occluded region of a body vessel. The filtering devices capture embolic material that may be created and released into the vessel during the procedure. The present invention is more particularly directed to an embolic filtering device made with a flexible and bendable expandable cage or basket. The concepts presented herein may be applied to any of a wide variety of other devices.

Numerous approaches have been developed for treating occluded blood vessels, usually involving the percutaneous introduction of an interventional device into the lumen of the artery, usually by a catheter. One widely known and medically accepted procedure is balloon angioplasty, in which an inflatable balloon is introduced within the stenosed region of the blood vessel to dilate the occluded vessel. The balloon dilatation catheter is initially inserted into the patient's arterial system and is advanced and manipulated into the area of stenosis in the artery. The balloon is inflated to compress the plaque and to press the vessel wall radially outward to increase the diameter of the blood vessel, resulting in increased blood flow. The balloon is then deflated to a small profile so that the physician can withdraw the dilatation catheter from the patient's vasculature. As should be appreciated by those skilled in the art, while the above-described procedure is typical, it is not the only method used in angioplasty.

Another procedure is laser angioplasty, which utilizes a laser to ablate the stenosis by super heating and vaporizing the deposited plaque. Still another method is atherectomy, in which cutting blades are rotated to shave the deposited plaque from the arterial wall. A catheter is usually used to capture the shaved plaque or thrombus from the bloodstream.

In the procedures of this kind, abrupt reclosure of the artery may occur, or restenosis of the artery may develop over time, requiring another angioplasty procedure, a surgical bypass operation, or some other method of repairing or strengthening the area. To reduce the likelihood of abrupt reclosure and to strengthen the area, a physician can implant an intravascular prosthesis for maintaining vascular patency, commonly known as a stent, inside the artery across the lesion. The stent can be crimped tightly onto the balloon portion of the catheter and transported in its delivery diameter through the patient's vasculature. At the deployment site, the stent is expanded to a larger diameter, often by inflating the balloon portion of the catheter. Alternatively, the stent may be of the self-expanding type, such that a balloon to expand the stent is not needed.

The above non-surgical interventional procedures, when successful, avoid the necessity of major surgical operations. However, there is one common problem associated with all of these non-surgical procedures, namely, the potential release of embolic debris into the bloodstream that can occlude distal vasculature and cause significant health problems to the patient. For example, pieces of plaque material are sometimes generated during a balloon angioplasty procedure and become released into the bloodstream. Or, during deployment of a stent, the metal struts of the stent may cut into the stenosis and shear off pieces of plaque that can travel downstream and lodge somewhere in the patient's vascular system. Also, while complete vaporization of plaque is the intended goal during laser angioplasty, sometimes particles are not fully vaporized and enter the bloodstream. Likewise, not all of the emboli created during an atherectomy procedure may be drawn into the catheter and, as a result, enter the bloodstream as well.

When any of the above-described procedures are performed in the carotid arteries, the release of emboli into the circulatory system can be extremely dangerous and sometimes fatal to the patient. Debris carried by the bloodstream to distal vessels of the brain can cause cerebral vessels to occlude, resulting in a stroke, and in some cases, death. Therefore, although cerebral percutaneous transluminal angioplasty has been performed in the past, the number of procedures performed has been somewhat limited due to the justifiable concern over an embolic stroke occurring should embolic debris enter the bloodstream and block vital downstream blood passages.

Medical devices have been developed to deal with the problem of debris or fragments entering the circulatory system following vessel treatment utilizing any one of the above-identified procedures. One approach that has been attempted is to cut any debris into minute sizes, which pose little chance of becoming occluded in major vessels within the patient's vasculature. However, it is often difficult to control the size of the fragments that are formed, and the potential risk of vessel occlusion still exists, making such a procedure in the carotid arteries a high-risk proposition.

Other techniques include the use of catheters with a vacuum source that provides temporary suction-to remove embolic debris from the bloodstream. There can be complications associated with such systems, however, if the vacuum catheter does not remove all of the embolic material from the bloodstream. Also, a powerful suction could cause trauma to the patient's vasculature.

Another technique that has had some success utilizes a filter or trap downstream or distal from the treatment site to capture embolic debris in the bloodstream. The placement of a filter in the patient's vasculature during treatment of the vascular lesion can reduce the presence of the embolic debris in the bloodstream. Such embolic filters are usually delivered in a collapsed position through the patient's vasculature and then expanded to trap the embolic debris. Some of these embolic filters are self expanding and utilize a restraining sheath which maintains the expandable filter in a collapsed position until it is ready to be expanded within the patient's vasculature. The physician can retract the proximal end of the restraining sheath to expose the expandable filter, causing the filter to expand at the desired location. Once the procedure is completed, the filter can be collapsed, and the filter (with the trapped embolic debris) can then be removed from the vessel.

Turning now to the structure of the embolic filter, in one popular design the distal and proximal ends of the struts of the expandable filter cage attach to respective sleeves. The process of attaching the strut ends onto the sleeve can be time consuming. One approach is for a technician to manually insert the strut ends in between inner and outer sleeves. Then the technician glues the strut ends into place, and cures the glue in an atmosphere of sufficient heat and/or humidity. The use of glue is generally thought to be superior to certain other approaches, such as welding. The heat from welding is typically believed to cause a change in material properties, such that the linear elastic nitinol material, from which the cage is cut, becomes superelastic. The device then does not perform in the manner for which it is designed.

But, there are problems with using glue, which tends to be messy and to flow to regions of the structure where glue is not desired. The manufacturing process is also less efficient than is desired, as the technician must normally use a microscope and have special skill in properly placing the tiny strut ends into a very small space between inner and outer sleeves. Also, glued joints generally do not have consistent strength, and a greater strength margin of safety is desired. This lack of joint strength may be the result of operator error, mating parts that are not entirely clean, accidental movement of the joint while the glue is curing, improper amount of glue applied, and/or the age of the glue. Another problem is that glue joints tend to be bulky, which is disadvantageous when attempting to reach small blood vessels.

What has been needed is a method of attaching the cage strut ends of the embolic filter to respective sleeves that avoids the messiness and inefficiency of a gluing process, but that does not adversely affect the desired material properties. The present invention disclosed herein satisfies these and other needs.

### SUMMARY OF THE INVENTION

The present invention provides an improved cage and sleeve assembly that is more efficient to manufacture. The assembly may be made to have a particularly small diameter that is advantageous for use in the body. The invention also includes a method for welding the ends of the cage struts together to form a sleeve, or for welding them onto a sleeve but, in any event, welding the ends so that the material properties in the regions of the cage that bend during use are not adversely affected by the heat of welding.

In one embodiment, a cage and sleeve assembly for an embolic filtering device used to filter embolic particles from a body vessel includes a strut assembly that is movable between an unexpanded position and an expanded position. The assembly includes struts that form a cage, with the struts. The strut ends are at least partially made of nitinol, and a series of welds secures the strut ends in the sleeve assembly.

This embodiment encompasses many variations. In one approach, spot welds join the ends of the struts to form a cylindrical tube, or even tube having a non-circular cross-section. This tube may be, for example, a sleeve that slides along a guidewire. Alternatively, the strut ends may be welded into place between an inner sleeve and an outer sleeve.

In another embodiment, a cage and sleeve assembly for an embolic filtering device used to filter embolic particles from a body vessel includes a nitinol strut assembly that is movable between an unexpanded position and an expanded position. Nitinol struts form a cage. A sleeve assembly includes the strut ends and a series of welds securing the strut ends to the sleeve assembly. The cage assembly includes heat affected zones and linear elastic zones The heat affected zones are confined to the strut ends, and do not extend into bending areas of the cage. For the welding, laser welding or spot welding is typically preferred, although other types of welding may be employed.

The invention includes a method of forming an embolic filter. The method includes laser cutting a nitinol hypotube into an embolic filter cage. Filter material is attached to at least a portion of the cage. The strut ends are welded within a heat affected zone. The cage also has linear elastic bending areas outside of the heat affected zone. The step of welding is carried out without causing material in the bending areas to become superelastic.

The method may optionally include other steps. For example, the strut ends may be inserted between an inner and an outer sleeve, and the strut ends welded to hold the strut ends in place in between the inner and outer sleeves. Alternatively, the method may include the steps of holding the strut in place on an inner sleeve using an outer sleeve, welding strut ends in place in between the inner and outer sleeves and then, after the welding step, removing the outer sleeve. Another method may include the step of welding includes welding strut ends together to form a sleeve.

It is to be understood that the present invention is not limited by the embodiments described herein. The present invention can be used in arteries, veins, and other body vessels. Other features and advantages of the present invention will become more apparent from the following detailed-description of the invention, when taken in conjunction with the accompanying exemplary drawings. The concept may also be extended beyond filtering devices, and encompass other devices as well.

### BRIEF DESCRIPTION OF THE DRAWINGS

FIG. 1 is a perspective view of an embolic filtering device with an expandable cage that is known in the art.

FIG. 2 is a perspective view of the expandable cage of FIG. 1 in its expanded configuration with the filter element removed to better show the expandable cage.

FIG. 3 is an elevational view, partially in cross section, of the embolic filtering device of FIG. 1 as it is being delivered within a body vessel downstream from an area to be treated.

FIG. 4 is an elevational view, partially in cross section, similar to that shown in FIG. 3, wherein the embolic filtering device is deployed in its expanded position within the body vessel for filtering purposes.

FIG. 5 is a perspective view of the strut ends at an end of a cage of an embolic filtering device.

FIG. 6 is a perspective view of a sleeve assembly at the proximal end of the embolic filtering device.

FIG. 7 is a perspective view of the strut ends of FIG. 5 mounted on a sleeve assembly at the distal end of the embolic filtering device.

FIG. 8 is a perspective view of one embodiment of a cage end assembly according to the present invention.

FIG. 9 is a perspective view of another embodiment of a cage end assembly according to the present invention.

FIG. 10 is a perspective view of another embodiment of a cage end assembly according to the present invention.

FIG. 11 illustrates an apparatus that may be used to implement an improved welding method according to the present invention.

### DETAILED DESCRIPTION OF THE PREFERRED EMBODIMENTS

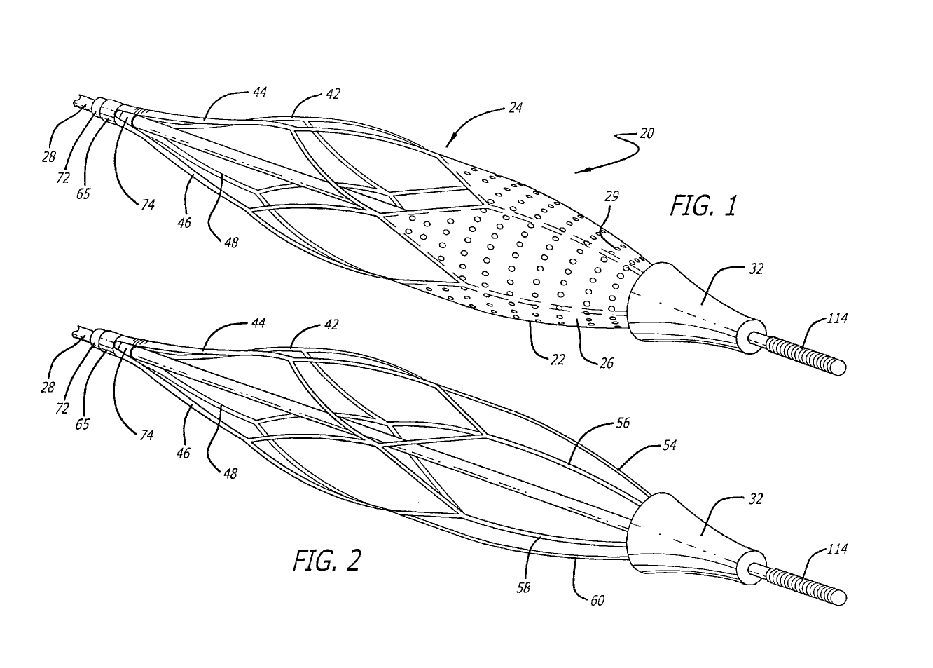

Turning now to the drawings, in which like reference numerals represent like or corresponding elements in the drawings, FIGS. 1 and 2 illustrate one particular embodiment of an embolic filtering device 20 that is known in the art. This embolic filtering device 20 is designed to capture embolic debris that may be created and released into a body vessel during an interventional procedure. The embolic filtering device 20 includes an expandable filter assembly 22 having a self-expanding basket or cage 24 and a filter element 26 attached thereto. In this particular embodiment, the expandable filter assembly 22 is rotatably mounted on the distal end of an elongated (solid or hollow) cylindrical tubular shaft, such as a guide wire 28.

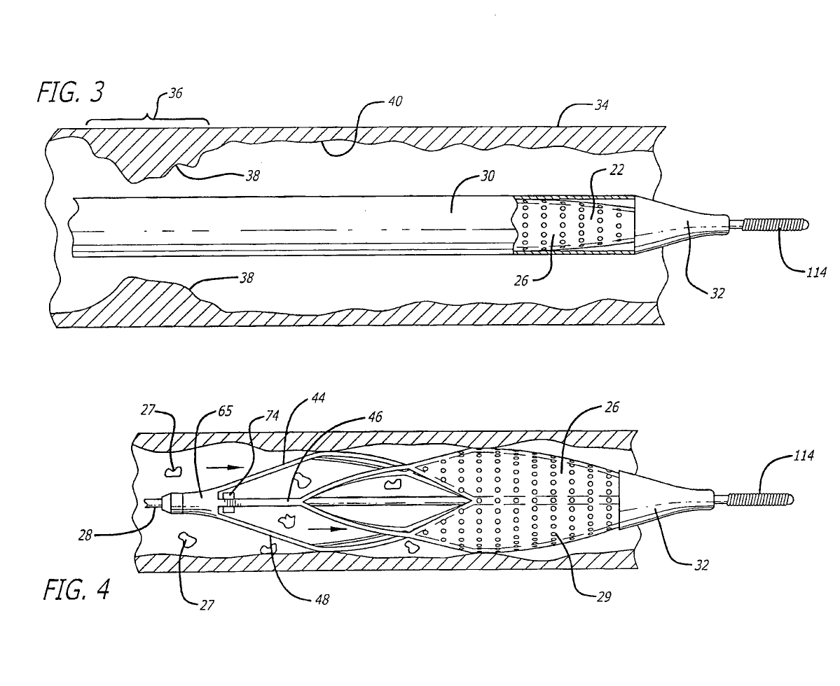

The guide wire has a proximal end (not shown) which extends outside the patient and is manipulated by the physician to deliver the filter assembly to the target lesion to be treated. A restraining or delivery sheath 30 (FIG. 3) extends coaxially along the guide wire 28 in order to maintain the expandable filter assembly 22 in its collapsed position until it is ready to be deployed within the patient's vasculature. The expandable filter assembly 22 is deployed by the physician by simply retracting the restraining sheath 30 proximally to expose the expandable filter assembly. As the restraining sheath is retracted, the self-expanding cage 24 immediately begins to expand within the body vessel (see FIG. 4), causing the filter element 26 to expand as well.

A pair of stop fittings 72 and 74 are placed on the guide wire to maintain the collar 65, and hence the proximal end of the expandable cage 24, rotatably fixed to the guide wire 28. These stop fittings 72 and 74 allow the expandable cage 24 to freely rotate on the guide wire while restricting the longitudinal movement of the cage on the guide wire. This particular mechanism is just one way in which the expandable cage 24 can be mounted to the guide wire 28. Alternatively, the expandable cage can be attached directly onto the guide wire so as not to rotate independently.

An obturator 32 affixed to the distal end of the filter assembly can be implemented to prevent possible “snowplowing” of the embolic filtering device as it is being delivered through the vasculature. The obturator can be made from a soft polymeric material, such as Pebax 40D, and has a smooth surface to help the embolic filtering device travel through the vasculature and cross lesions while preventing the distal end of the restraining sheath 30 from “digging” or “snowplowing” into the wall of the body vessel.

In FIGS. 3 and 4, the embolic filtering device 20 is shown as it is being delivered within an artery 34 or other body vessel of the patient. In FIG. 4, the embolic filtering device 20 is shown in its expanded position within the patient's artery 34. The artery (FIG. 3) has an area of treatment 36 in which atherosclerotic plaque 38 has built up against the inside wall 40 of the artery 34. The filter assembly 22 is to be placed distal to, and downstream from, the area of treatment 36. For example, the therapeutic interventional procedure may comprise the implantation of a stent (not shown) to increase the diameter of an occluded artery and increase the flow of blood therethrough.

It should be appreciated that the embodiments of the embolic filtering device described herein are illustrated and described by way of example only and not by way of limitation. Also, while the present invention is described in detail as applied to an artery of the patient, those skilled in the art will appreciate that it can also be used in other body vessels, such as the coronary arteries, carotid arteries, renal arteries, saphenous vein grafts and other peripheral arteries, as well as veins, the pulmonary system and other channels for bodily fluid or gas. Additionally, the present invention can be utilized when a physician performs any one of a number of interventional procedures, such as balloon angioplasty or atherectomy which generally require an embolic filtering device to capture embolic debris created during the procedure.

The cage 24 includes self-expanding struts which, upon release from the restraining sheath 30, expand the filter element 26 into its deployed position within the artery (FIG. 4). Embolic particles 27 created during the interventional procedure and released into the bloodstream are captured within the deployed filter element 26. The filter may include perfusion openings 29, or other suitable perfusion means, for allowing blood flow through the filter 26.

The filter element will capture embolic particles that are larger than the perfusion openings while allowing some blood to perfuse downstream to vital organs. Although not shown, after the deployment of the filter, a balloon angioplasty catheter can be initially introduced within the patient's vasculature in a conventional Seldinger technique through a guiding catheter (not shown). The guide wire 28 is disposed through the area of treatment and the dilatation catheter can be advanced over the guide wire 28 within the artery 34 until the balloon portion is directly in the area of treatment 36. The balloon of the dilatation catheter can be expanded, expanding the plaque 38 against the wall 40 of the artery 34 to expand the artery and reduce the blockage in the vessel at the position of the plaque 38. After the dilatation catheter is removed from the patient's vasculature, a stent (not shown) could be implanted in the area of treatment 36 using over-the-wire or rapid exchange techniques to help hold and maintain this portion of the artery 34 and help prevent restenosis from occurring in the area of treatment.

The stent could be delivered to the area of treatment on a stent delivery catheter (not shown) that is advanced from the proximal end of the guide wire to the area of treatment. Any embolic debris created during the interventional procedure will be released into the bloodstream and should be captured by the filter 26. Once the procedure is completed, the interventional device may be removed from the guide wire. The filter assembly 22 can also be collapsed and removed from the artery 34, taking with it any embolic debris trapped within the filter element 26. A recovery sheath (not shown) can be delivered over the guide wire 28 to collapse the filter assembly 22 for removal from the patient's vasculature.

Referring again to FIGS. 1 and 2, the expandable cage 24 includes four self-expanding proximal struts 42-48. These struts help to deploy the filter element 26 and the remainder of the expandable cage. Similarly, four distal struts 54-60 extend distally towards the obturator 32. These struts also aid in expanding the cage.

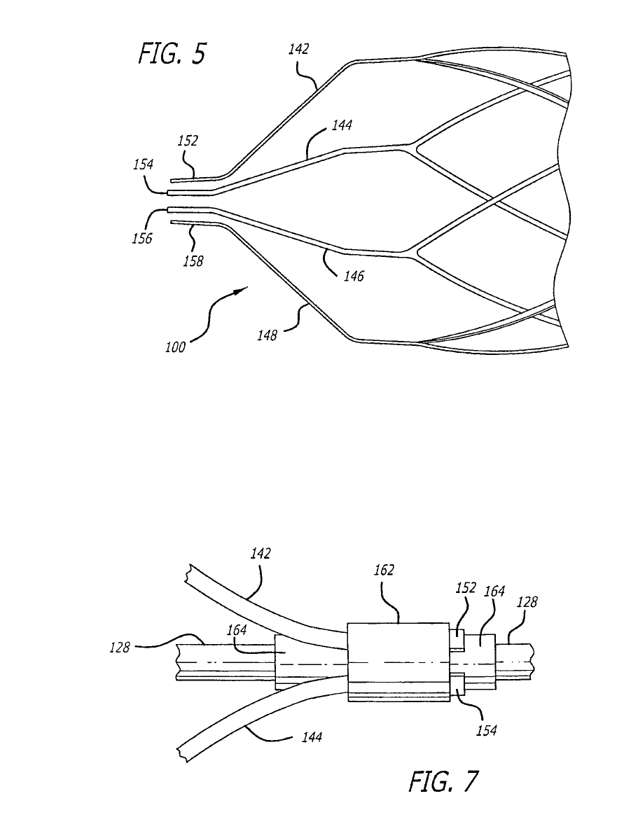

Referring now to FIG. 5, the expandable cage 24 is shown as it appears after it has been laser cut from a tubular member and strut ends 152, 154, 156 and 158 on struts 142, 144, 146 and 148 are mechanically bent to a smaller diameter. As can be seen, the free ends of the proximal and distal struts are initially spaced apart after being formed from the tubular member. The free ends of the struts can be attached to a collar, such as is shown in FIGS. 1 and 2, to allow the expandable cage to be mounted to an elongated member, such as a guide wire.

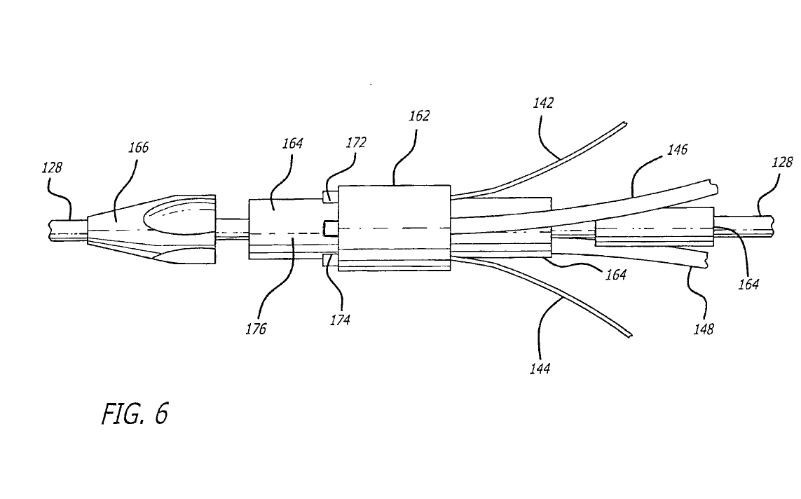

As discussed above, a known method of attaching the free ends of the proximal and distal struts to the collar with glue. FIG. 5 illustrates a cage frame that has been cut from a thin-walled tube of nitinol using a laser cutter, according to methods known in the art. The cage frame has four proximal struts: 142, 144, 146 and 148. These proximal struts are all part of the structure that forms the proximal end 100 of the embolic filter. At the end of each of the struts, there is a strut end, which is used to attach the cage to one or more related proximal sleeves. One such proximal sleeve assembly is illustrated in FIG. 6, in which an inner sleeve 164 and an outer sleeve 162 form a structure into which the associated strut ends may be inserted and held in place.

In the arrangement of FIG. 6, each of the struts 142, 144, 146 and 148 has its associated strut end inserted in between the inner sleeve 164 and the outer sleeve 162. In a present method of manufacture, a technician manually inserts the strut ends 152-158 in between the inner sleeve 164 and the outer sleeve 162. As these parts are very tiny, the technician typically must use a microscope and must have considerable skill and agility to thread the strut ends 152-158 in between the inner and outer sleeves. Oftentimes, the technician must spend training time to learn how to perform this procedure. Also, because a microscope must be used, the procedure can be somewhat time consuming.

Once the technician has inserted the strut ends 152-158 in between the inner and outer sleeve 162, 164, the technician typically glues the strut ends into place. Working with glue can have drawbacks. First, after the glue is applied, the glue must then be cured. One common method of curing the glue is to insert the assembly inside a chamber in which the humidity is relatively high. When a water-activated adhesive is utilized, the humidity in the chamber will activate the glue. The glue hardens and the strut ends are set into place. While many high quality embolic filters have been manufactured utilizing this approach, nevertheless the process can be time consuming and sometimes messy, particularly when the glue migrates to areas where glue is not desired. Other disadvantages of glue joints have been discussed previously.

Considering other elements of the structure illustrated in FIG. 6, the embolic filter is mounted, ultimately, on a guidewire 128. The inner sleeve 164, together with the outer sleeve 162 and the strut ends 152-158, may slide longitudinally along the guidewire 128. Stop 166 at the proximal side and stop 168 at the distal side limit the distance that the inner sleeve 164 can travel. But the inner cylinder 164 is typically free to rotate about the guidewire 128, which assists the physician when the filter is deployed, particularly when the physician needs to twist the guidewire during the procedure.

FIG. 7 is a close-up view of an alternative embodiment in which glue alone is used to ensure that the strut ends do not migrate from in between the outer sleeve 162 and the inner sleeve 164. The arrangement of FIG. 7 may be used advantageously on the distal end of the embolic filter, for example, where the stresses on the struts 142, 144 and the like tend to be less than on the proximal end of the filter. The proximal joints experience more stress since delivery and recovery of the filter is affected by forces transferring from the wire through the joints, to move the filter against frictional forces in the delivery or recovery sheath.

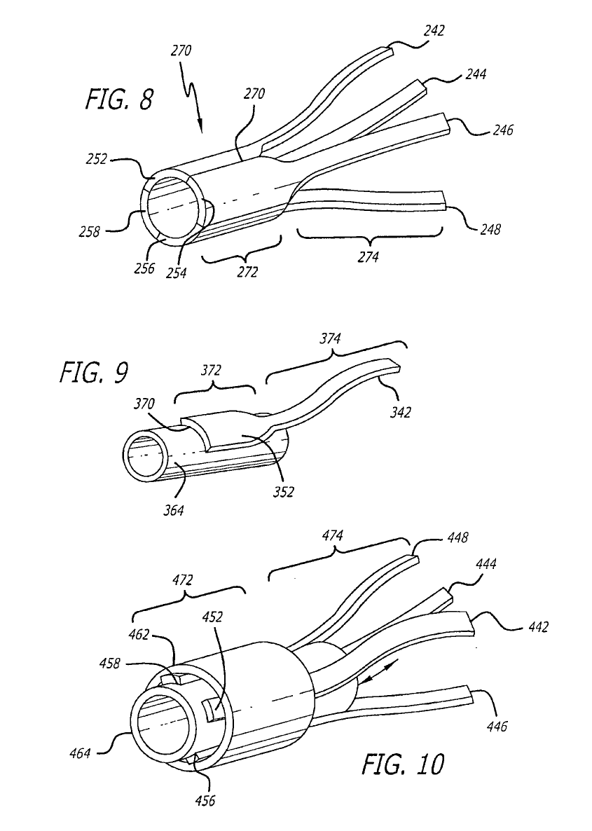

Considering now one aspect of the present invention, FIG. 8 illustrates an embodiment in which gluing is not required. Also, the diameter of the sleeve assembly is reduced, because no inner and outer sleeves such as 164 and 162 of FIG. 7, are required in this embodiment. With a reduced diameter, the insertion sleeve, also known as delivery sheath 3 (FIG. 3), may have a smaller diameter. This improves performance of the entire guidewire assembly within the body, such that the physician may more easily maneuver the assembly within the body. Also, the filter can be delivered through smaller passages in partially occluded vessels and delivered to vessels of smaller diameter.

FIG. 8 shows a series of cage struts 242, 244, 246 and 248. At the end of the struts are respective strut ends 252, 254, 256 and 258. The strut ends each have a partially cylindrical cross-section. This can be accomplished during the step of cutting the cage, since the cage is typically cut from a single cylinder of material.

The strut ends 252-258 are welded together to form a cylinder. This cylinder acts as something similar to the inner sleeve 164 of FIG. 7. That is, a sleeve 264 is formed directly from the strut ends 252-258. The strut ends are welded together along the seams formed along adjacent strut ends.

It is important that during the welding stage, heat is not allowed to migrate outside of the area of the welds. This is because the high temperature of the welds will transform the nitinol material from linear elastic behavior to superelastic behavior. It is intended that the struts 242-248 bend in a linearly elastic fashion. Consequently, it is important to prevent heat from building up in the bending area of the struts to a degree such that the linear elastic material becomes superelastic. To prevent the heat from migrating outside of the heat affected zone 272, a low power laser welder. In addition, a heat sink in the form of a copper cylinder that temporarily goes inside of the cylinder 264, may be employed to conduct heat away from the heat affected zone during welding. This permits the linear elastic zone 274 to remain linear elastic, and the nitinol in the linear elastic zone 274 is not transformed into a superelastic material.

Another alternative embodiment is illustrated in FIG. 9. In the embodiment of FIG. 9, the strut 342 has a strut end 352 that is partially cylindrical in profile. This strut end 352 is spot or laser welded directly onto an inner sleeve 364. In this way, no outer sleeve, such as sleeve 162 of FIG. 7, is necessary. The heat affected zone remains within area 372, whereas the working portion of the cage assembly that is bent remains in a linear elastic zone 374. In FIG. 9, although only strut end 352 is shown, in practice there will be multiple strut ends, each corresponding to an end of a cage strut.

One advantage of the approach of FIG. 9 is that, again, an outer sleeve 162 (FIG. 7) is not needed. Consequently, the diameter of the sleeve assembly is thereby reduced, and a smaller diameter insertion sheath may be used. This has advantages to the physician during use of the embolic filter assembly, particularly while the assembly is being inserted into the body.

Considering the embodiment of FIG. 10, both an inner sleeve 464 and an outer sleeve 462 are utilized. However, no glue is necessary. The strut ends 452-458 are spot or laser welded at the ends. Laser and spot welding are techniques generally known in the art. In laser welding, two pieces of material to be welded are placed in close proximity. A laser beam is directed at these adjacent materials. The materials heat up, melt and fuse together as they cool. In spot welding, two pieces of material to be welded are clamped together between two electrodes. A current is passed between the two electrodes and through the two materials. Electrical resistance between the two materials causes heat generation that melts the two materials in a spot between the two electrodes. The current is stopped, the material cools and fuses together. The clamping electrodes are then removed. The welding can be done with a welding apparatus known in the art. Welding methods other than spot welding and laser welding can be used.

Since the strut ends are welded only at the ends, the heat affected zone 472 does not extend into the region in which bending normally takes place. That is, the linear elastic zone 474 remains linear elastic and the bending area can perform as desired. Because the heat affected zone 472 is limited to the region near where the ends of the strut ends are welded, only the nitinol in the heat affected zone 472 becomes superelastic. The nitinol in the linear elastic zone 474 is not heated sufficiently to turn that nitinol into superelastic material.

Although the embodiment of FIG. 10 does use an inner and outer sleeve, as in the embodiment of FIG. 7, there is nevertheless an advantage in that the embodiment of FIG. 10 does not utilize any glue. The steps of gluing and curing the glue are eliminated. This tends to increase efficiency of manufacture, and reduces the disadvantages of using glue, which can become messy. If glue were to be used, curing time to stabilize the joint would not be needed before proceeding on to the next step of manufacture. In addition, unlike glue, each strut end could be quickly secured by a weld rather than being held in place while the glue cures.

In a related approach, the outer sleeve 472 may be used solely during manufacture for the purpose of holding the strut ends 452-458 in place during welding. Then, when welding is finished, the outer sleeve 462 may be removed. In this approach, the diameter of the assembly is thereby reduced, because the outer sleeve 462 does not remain on the assembly as it is inserted into the body. It is noted, however, that this embodiment with the outer sleeve 462 being removed is just one approach. In another approach, the sleeve 462 remains in place and may even be welded onto the strut ends 452-458 to hold the outer sleeve 462 in place.

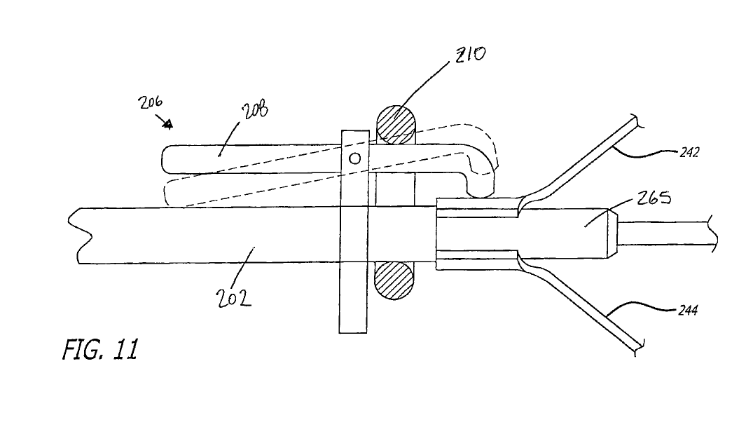

FIG. 11 illustrates an example of an apparatus 200 that may be used to weld ends of strut ends 242, 244 onto a sleeve 265. A sleeve 265 is mounted on a mandrel 202 made of brass or anodized aluminum. The mandrel 202 may include a step 204 to aid in aligning the ends of the strut ends.

The mandrel 202 acts as a heat sink to carry away heat from the sleeve 265 during welding. A clamp arm 206 having a clamp handle 208 is pivotally mounted so as to pivot onto an end of a cage strut end. The clamp arm 206 may be inserted underneath an O-ring 210, to spring load the clamp. The clamp also acts as a heat conductor that helps to carry heat away from the strut end being welded.

In use, a technician pushes the handle 208 toward the mandrel and positions an end of a strut end onto the sleeve 265. The technician then releases the handle so that the strut end is held into place. The laser welding apparatus (not shown) may then be operated, to weld the strut end onto the sleeve. The mandrel 202 and the clamp arm 206 act as heat conductors that help carry heat away from the strut end during welding, thereby limiting the extent of the heat-affected zone and helping to prevent welding heat from transforming material in bending regions of the strut end.

In FIG. 11, only one clamp arm 206 is shown. However, in practice, one clamp arm may be supplied for each of the corresponding strut ends. Alternatively, a single clamp arm may be used, and the mandrel 202 rotated after each strut end is welded into place, for sequential welding. This process could also be used for the FIG. 8 and/or FIG. 10 designs where strut ends are welded together.

The expandable cage of the present invention can be made in many ways. One particular method is to cut a thin-walled tubular member, such as nickel-titanium hypotube, to remove portions of the tubing in the desired pattern for each strut, leaving relatively untouched the portions of the tubing which form each strut. The tubing may be cut into the desired pattern by means of a machine-controlled laser. Prior to laser cutting the strut pattern, the tubular member could be formed with varying wall thicknesses which will be used to create the flexing portions of the cage.

The tubing used to make the cage could possibly be made of suitable biocompatible material such as spring steel. Elgiloy® is another material that could possibly be used to manufacture the cage. Also, very elastic polymers could be used to manufacture the cage.

The strut size is often very small, so the tubing from which the cage is made must necessarily have a small diameter. Typically, the tubing has an outer diameter of that of the final expanded cage and of the order of a few millimeters. Linear elastic tubing generally is lased full cage size tubing, and not expanded from a smaller size. Self expanding stents of super elastic material are lased from small tubing and then mechanically set or heat set to a larger size. The wall thickness of the tubing is usually only a fraction of a millimeter. For cages implanted in body lumens, such as PTA applications, the dimensions of the tubing may be correspondingly larger.

While it is preferred that the cage be made from laser cut tubing, those skilled in the art will realize that the cage can be laser cut from a flat sheet and then rolled up in a cylindrical configuration with the longitudinal edges welded to form a cylindrical member. Also, the cage may be cut by other methods known in the art. If welded, the welding areas can be confined to non-bending areas of the cage so that the bending areas are outside the welded heat-effected zones.

Generally, the tubing is put in a rotatable collet fixture of a machine-controlled apparatus for positioning the tubing relative to a laser. According to machine-encoded instructions, the tubing is then rotated and moved longitudinally relative to the laser which is also machine-controlled. The laser selectively removes the material from the tubing by ablation and a pattern is cut into the tube. The tube is therefore cut into the discrete pattern of the finished struts. The cage can be laser cut much like a stent is laser cut. Details on how the tubing can be cut by a laser are found in U.S. Pat. No. 5,759,192 (Saunders), U.S. Pat. No. 5,780,807 (Saunders) and U.S. Pat. No. 6,131,266 (Saunders) which have been assigned to Advanced Cardiovascular Systems, Inc.

The process of cutting a pattern for the strut assembly into the tubing generally is automated, except for loading and unloading the length of tubing. For example, a pattern can be cut in tubing using a CNC-opposing collet fixture for axial rotation of the length of tubing, in conjunction with CNC X/Y table to move the length of tubing axially relative to a machine-controlled laser as described. The entire space between collets can be patterned using the CO₂ or Nd:YAG laser set-up. The program for control of the apparatus is dependent on the particular configuration used and the pattern to be ablated in the coding.

A suitable composition of nickel-titanium that can be used to manufacture the strut assembly of the present invention is approximately 55% nickel and 45% titanium (by weight) with trace amounts of other elements making up about 0.5% of the composition. The upper plateau strength is about a minimum of 60,000 psi with an ultimate tensile strength of a minimum of about 155,000 psi. The permanent set (after applying 8% strain and unloading), is approximately 0.5%. The breaking elongation is a minimum of 10%. It should be appreciated that other compositions of nickel-titanium can be utilized, as can other self-expanding alloys, to obtain the features of a self-expanding cage made in accordance with the present invention. That is, this is only one example of a suitable material, and other suitable material compositions known in the art or developed in the future may be used within the scope of the invention.

The cage can also be manufactured by laser cutting a large diameter tubing of nickel-titanium which would create the cage in its expanded position. Thereafter, the formed cage could be placed in its unexpanded position by backloading the cage into a restraining sheath which will keep the device in the unexpanded position until it is ready for use.

In an alternative embodiment, the struts of the proximal strut assembly can be made from a different material than the distal strut assembly. In this manner, more or less flexibility for the proximal strut assembly can be obtained. When a different material is utilized for the struts of the distal proximal strut, the distal strut assembly can be manufactured through the process described above, with the struts of the proximal strut assembly being formed separately and attached to the distal assembly. Suitable fastening means such as adhesive bonding, brazing, soldering, welding and the like can be utilized in order to connect the proximal struts to the distal assembly. Suitable materials for the struts include materials such as nickel-titanium, spring steel, Elgiloy®, along with polymeric materials which are sufficiently flexible and bendable.

Polymeric materials that can be utilized to create the filtering element include, but are not limited to, polyurethane, Gortex®, and ePTFE. The material can be elastic or non-elastic. The wall thickness of the filtering element is typically about 0.00050-0.0050 inches, although the wall thickness may vary depending on the particular material selected. The material can be made into a cone or similarly sized shape utilizing blow-mold or dip molding technology. The openings can be any different shape or size. A laser, a heated rod or other process can be utilized to create to perfusion openings in the filter material. The holes, would of course be properly sized to catch the particular size of embolic debris of interest.

The materials which can be utilized for the restraining sheath can be made from polymeric material such as cross-linked HDPE. This sheath can alternatively be made from a material such as polyolifin which has sufficient strength to hold the compressed strut assembly and has relatively low frictional characteristics to minimize any friction between the filtering assembly and the sheath. Friction can be further reduced by applying a coat of silicone lubricant, such as Microglide®, to the inside surface of the restraining sheath before the sheaths are placed over the filtering assembly.

Regarding terminology, the term “tube” in the claims is not limited to circular cross-sections, but includes other closed cross-sections that may be useful in the context of this type of device, including square, triangular, or elliptical cross-sections and the like.

Further modifications and improvements may additionally be made to the device and method disclosed herein without departing from the scope of the present invention. Accordingly, it is not intended that the invention be limited, except as by the appended claims.

What is claimed is:

1. A cage and sleeve assembly for an embolic filtering device used to filter embolic particles from a body vessel, comprising:
a strut assembly that is movable between an unexpanded position and an expanded position;
a plurality of struts forming a cage, the struts having strut ends at the respective ends; and
a sleeve assembly;
wherein the strut ends comprise nitinol, and wherein the sleeve assembly comprises the strut ends and a series of welds securing the strut ends in the sleeve assembly.

2. A cage and sleeve assembly as defined in claim 1, wherein the strut ends comprise ends, and the welds join the ends to form a tube.

3. A cage and sleeve assembly as defined in claim 1, wherein the tube has a circular cross-section.

4. A cage and sleeve assembly as defined in claim 1, wherein the tube has a non-circular cross-section.

5. A cage and sleeve assembly as defined in claim 1, wherein the welds join ends of the strut ends to form a tube that is adapted to slide and rotate along a guidewire.

6. A cage and sleeve assembly as defined in claim 1, wherein the strut ends are partially cylindrical, and welds join strut end ends together to form a cylindrical sleeve.

7. A cage and sleeve assembly as defined in claim 1, wherein the strut ends are welded onto a sleeve.

8. A cage and sleeve assembly as defined in claim 1, wherein strut ends are welded onto a sleeve only at end tips of the strut ends.

9. A cage and sleeve assembly as defined in claim 1, wherein strut ends have ends that are partial cylinders, and the partial cylinders are welded onto a cylindrical sleeve.

10. A cage and sleeve assembly as defined in claim 1, wherein strut ends are welded into place between an inner sleeve and an outer sleeve.

11. A cage and sleeve assembly as defined in claim 1, wherein the cage struts and strut ends are nitinol.

12. A cage and sleeve assembly for an embolic filtering device used to filter embolic particles from a body vessel, comprising:
a nitinol strut assembly that is movable between an unexpanded position and an expanded position;
a plurality of nitinol struts forming a cage, the struts having nitinol strut ends at the respective ends; and
a sleeve assembly;
wherein the sleeve assembly comprises the strut ends and a series of welds securing the strut ends to the sleeve assembly; and
wherein the cage assembly includes heat affected zones and linear elastic zones, the heat affected zones being confined to the strut ends and not extending into bending areas of the cage.

13. A cage and sleeve assembly as defined in claim 12, wherein the strut ends comprise ends, and the welds join the ends to form a tube.

14. A cage and sleeve assembly as defined in claim 12, wherein the tube has a circular cross-section.

15. A cage and sleeve assembly as defined in claim 12, wherein the tube has a non-circular cross-section.

16. A cage and sleeve assembly as defined in claim 12, wherein the welds join ends of the strut ends to form a tube that is adapted to slide and rotate along a guidewire.

17. A cage and sleeve assembly as defined in claim 12, wherein the strut ends are partially cylindrical, and welds join strut end ends together to form a cylindrical sleeve.

18. A cage and sleeve assembly as defined in claim 12, wherein the strut ends are welded onto a sleeve.

19. A cage and sleeve assembly as defined in claim 12, wherein strut ends are welded onto a sleeve only at end tips of the strut ends.

20. A cage and sleeve assembly as defined in claim 12, wherein strut ends have ends that are partial cylinders, and the partial cylinders are welded onto a cylindrical sleeve.

21. A cage and sleeve assembly as defined in claim 12, wherein strut ends are welded into place between an inner sleeve and an outer sleeve.

22. A method of forming an embolic filter as defined in claim 12, comprising the steps of:
laser cutting a nitinol hypotube into an embolic filter cage;
attaching filter material to at least a portion of the cage;
forming strut ends at strut ends of the cage;
welding the strut ends within a heat affected zone, the cage having linear elastic bending areas outside of the heat affected zone, the step of welding being carried out without causing material in the bending areas to become superelastic.

23. A method of forming an embolic filter as defined in claim 22, wherein the method includes the step of inserting strut ends between an inner and an outer sleeve, welding the strut ends to hold the strut ends in place in between the inner and outer sleeves

24. A method of forming an embolic filter as defined in claim 22, wherein the method includes the step of inserting strut ends between an inner and an outer sleeve and welding strut ends onto a sleeve.

25. A method of forming an embolic filter as defined in claim 22, wherein the method includes the steps of holding the strut in place on an inner sleeve using an outer sleeve, welding strut ends in place in between the inner and outer sleeves and then, after the welding step, removing the outer sleeve.

26. A method of forming an embolic filter as defined in claim 22, wherein the step of welding includes welding strut ends together to form a sleeve.

27. A method of forming an embolic filter as defined in claim 22, wherein the sleeve has a curvature, and the strut ends have a curved profile that mates with the curvature of the sleeve, and the strut ends are welded onto the sleeve.
